(19) United States
(12) Patent Application Publication
(10) Pub. No.: US 2023/0077692 A1
(43) Pub. Date: Mar. 16, 2023

(54) MECHANISM FOR REDUCING INFORMATION LOST IN SET NEURAL NETWORKS

(71) Applicant: NEC Laboratories Europe GmbH, Heidelberg (DE)

(72) Inventors: Daniel Onoro-Rubio, Heidelberg (DE); Francesco Alesiani, Heidelberg (DE)

(21) Appl. No.: 17/560,322

(22) Filed: Dec. 23, 2021

Related U.S. Application Data
(60) Provisional application No. 63/244,754, filed on Sep. 16, 2021.

(51) Int. Cl. G06N 3/08

(52) U.S. Cl. CPC G06N 3/08

(57) ABSTRACT

A method for minimizing information loss in set neural networks includes determining an information loss term for a set neural network that internally uses virtual tokens, such that the information loss term minimizes a divergence between two distributions. The set neural network is trained with training data from a data source that is expressed as sets using the information loss term.

### CROSS-REFERENCE TO RELATED APPLICATION

Priority is claimed to U.S. Provisional Patent Application No. 63/244,754, filed on Sep. 16, 2021, the entire disclosure of which is hereby incorporated by reference herein.

### FIELD

The present invention relates to artificial intelligence (AI) and machine learning (ML), and in a particular method, system, and computer-readable medium for reducing information lost in set neural networks.

### BACKGROUND

Learning representations to model sets is a problem that has recently gained attention. Pioneer works such as the Graph Convolutional Networks were used to learn representations on arbitrary graph structures (see, e.g., Defferrard, et al., “Convolutional Neural Networks on Graphs with Fast Localized Spectral Filtering,” 30th Conference on Neural Information Processing Systems (NIPS 2016), Barcelona, Spain, arXiv: 1606.09375v3 [cs.LG] 5 Feb. 2017, which is hereby incorporated by reference herein). This opened the door to models like PointNet that learn models for 3D point cloud object classification and segmentation (see, e.g., Qi, et al., “PointNet: Deep Learning on Point Sets for 3D Classification and Segmentation,” CVPR. 2017, arXiv: 1612.00593v2 [cs.CV] 10 Apr. 2017, which is hereby incorporated by reference herein). Meanwhile, self-attention mechanisms and transformer models gained popularity. As the result, very popular language models such as Bidirectional Encoder Representations from Transformers (BERT) appeared for sentence classification (see, e.g., Devlin, et al., “BERT: Pre-training of Deep Bidirectional Transformers for Language Understanding,” arXiv: 1810.04805v2 [cs.CL] 24 May 2019, which is hereby incorporated by reference herein). BERT incorporates a classification token to the model used for classifying a whole sentence. Then, following this trend there was provided the set-transformer (see, e.g., Lee, et al., “Set Transformer: A Framework for Attention-based Permutation-Invariant Neural Networks,” Proceedings of the 36^{th} International Conference on Machine Learning, Long Beach, Calif., PMLR 97, 2019., arXiv: 1810.00825v3 [cs.LG] 26 May 2019, which is hereby incorporated by reference herein).

### SUMMARY

An embodiment of the present invention provides a method for minimizing information loss in set neural networks. The method includes determining an information loss term for a set neural network that internally uses virtual tokens, such that the information loss term minimizes a divergence between two distributions, and training the set neural network with training data from a data source that is expressed as sets using the information loss term.

### BRIEF DESCRIPTION OF THE DRAWINGS

Subject matter of the present disclosure will be described in even greater detail below based on the exemplary figures. All features described and/or illustrated herein can be used alone or combined in different combinations. The features and advantages of various embodiments will become apparent by reading the following detailed description with reference to the attached drawings, which illustrate the following:

FIG. 1 illustrates an exemplary embodiment of a set transformer;

FIG. 2a illustrates a t-distributed stochastic neighbor embedding (tSNE) projection learned with a plain model of virtual tokens and input tokens of an exemplary real input for a fingerprint dataset;

FIG. 2b illustrates a tSNE projection learned with an embodiment of the present invention of virtual tokens and input tokens of the exemplary real input for a fingerprint dataset;

FIG. 3 illustrates how an input is fed into a model according to an exemplary embodiment of the present invention and how losses are connected during a training time;

FIG. 4 shows a ranking progression of a validation over time during training;

FIG. 5 illustrates an exemplary embodiment of the present invention as applied to a fingerprint system;

FIG. 6 illustrates an exemplary embodiment of the present invention for listing possible binding proteins; and

FIG. 7 illustrates an exemplary embodiment of the present invention applied to robotics, in which a robot has to identify objects, and interact with them.

### DETAILED DESCRIPTION

In the model of the set-transformer, it was proposed to use a collection of virtual tokens that are used to internally encode an input set. However, during the training, the models are optimized to solve the main target task, and despite the relatively good performance of the model, there is an information loss between the input and the virtual tokens that are used for the encoding.

Embodiments of the present invention provide a system, method, and computer-readable medium for minimizing information loss during the encoding of minutiae sets. The system can operate on data expressed as sets, and to set models that internally make use of collections of virtual tokens. The method utilizes the minimization of divergence between two distributions. In particular, embodiments of the present invention improve methods and computer systems for the training of set neural networks such as, for example, set-transformers, by solving the technical problem of information loss in encoding. According to embodiments of the present invention, a mechanism in the form of an information loss term that is added to the training acts to reduce the information loss and can render set neural networks able to be trained using any conventional algorithm. The method according to embodiments of the present invention provides improvements in the form of having a direct impact to increase the convergence speed during the training, increasing the generalization capability of the model, and improving the final performance of the model. This results in savings in computational resources and costs by reducing the energy and time that is required for training the model, while at the same time improving performance of the final system. Moreover, embodiments of the present invention reduce the need for retraining the model due to the improved generalization, resulting in further savings in computational resources, time, and costs, while simultaneously increasing the flexibility and applicability of the final system. Further, devices that interact with the present invention's output, such as simple computing units, can experience improved functioning by acquiring quick and offline results on the edge.

Embodiments of the present invention provide a system, method, and computer-readable medium for efficiently training set neural networks. The method according to an embodiment comprises the steps of accessing a data source that can be expressed as sets, a training dataset, and a set of neural networks that internally use virtual tokens; training the neural network with the proposed information loss term [symbol] _{Vt} for the minimization of the divergence between two distributions with metrics such as Kullback-Leibler (KL) divergence (preferably), or others; and testing and deploying the method.

The system according to an embodiment of the present invention comprises one or more hardware processors having access to physical memory which configures the processors to be able to execute a method according to an embodiment of the present invention.

The computer-readable medium according to an embodiment of the present invention is tangible and non-transitory and contains computer-executable instructions which, upon being executed by one or more processors, facilitate execution of a method according to an embodiment of the present invention.

In an embodiment, the method for training set neural networks is improved through the addition of an information loss term, wherein the addition minimizes the information loss of the encoding performed by the virtual tokens and the input during the training in a set neural network.

In an embodiment, the method for training set neural networks minimizes the divergence between two distributions with metrics such as KL divergence or other preferred metrics.

The method for training set neural networks may, in various other embodiments, provide methods for matching the minutiae of fingerprints, predicting protein-to-protein binding based on representations of a molecule as a set of 3 dimensional (3D) points, and assisting a robotic device, such as an arm, identify and interact with objects in dimensional space.

Aspect (1): In an aspect (1), the present invention provides a method for minimizing information loss in set neural networks. The method comprises determining an information loss term for a set neural network that internally uses virtual tokens, such that the information loss term minimizes a divergence between two distributions, and training the set neural network with training data from a data source that is expressed as sets using the information loss term.

Aspect (2): In an aspect (2), the present invention provides the method according to aspect (1), wherein minimization of the divergence is performed using a metric that measures the divergence between a distribution of the virtual tokens and a distribution of input tokens.

Aspect (3): In an aspect (3), the present invention provides the method according to the aspects (1) or (2), wherein the metric is a Kullback-Leibler divergence, Wasserstein, or Jensen-Shannon divergence.

Aspect (4): In an aspect (4), the present invention provides the method according to the aspects (1), (2), or (3), wherein the aspect further includes testing the trained set neural network.

Aspect (5): In an aspect (5), the present invention provides the method according to the aspects (1), (2), (3), or (4), wherein the aspect further comprises using the trained set neural network to produce a compressed representation of input data in a machine learning task.

Aspect (6): In an aspect (6), the present invention provides the method according to the aspects (1), (2), (3), (4), or (5), wherein the aspect further comprises obtaining minutiae of fingerprints as the training data from the data source that is expressed as sets, and encoding in a compressed representation the minutiae of fingerprints.

Aspect (7): In an aspect (7), the present invention provides the method according to the aspects (1), (2), (3), (4), (5), or (6), wherein the method uses the compressed representation to match a fingerprint.

Aspect (8): In an aspect (8), the present invention provides the method according to the aspects (1), (2), (3), (4), (5), (6), or (7), wherein the method depicts a protein molecule as a set of 3D points as the data source that can be expressed as sets, and uses the trained set neural network to predict a protein binding candidate from a protein representation dataset.

Aspect (9): In an aspect (9), the present invention provides the method according to the aspects (1), (2), (3), (4), (5), (6), (7), or (8), wherein the method defines an object by a set of 3D points as the training data from the data source that is expressed as sets, and uses the trained set neural network to classify the object into an object class.

Aspect (10): In an aspect (10), the present invention provides the method according to the aspects (1), (2), (3), (4), (5), (6), (7), (8), or (9), wherein the set neural network internally uses mean and variance of the virtual tokens during the training.

Aspect (11): In an aspect (11), the present invention provides the method according to the aspects (1), (2), (3), (4), (5), (6), (7), (8), (9), or (10), wherein the aspects encode a compressed representation of data of the data source that is expressed as sets using the trained set neural network.

Aspect (12): In an aspect (12), the present invention provides the method according to the aspects (1), (2), (3), (4), (5), (6), (7), (8), (9), (10), or (11), wherein the divergence is between a distribution of virtual tokens and a distribution of input tokens and the divergence approximates an input token space.

Aspect (13): In an aspect (13), the present invention provides a system including one or more hardware processors which, alone or in combination, are configured to provide for execution of the steps of determining an information loss term for a set neural network that internally uses virtual tokens such that the information loss term minimizes a divergence between two distributions, and training the set neural network with training data from a data source that is expressed as sets using the information loss term.

Aspect (14): In an aspect (14), the present invention provides the system according to the aspect (13), wherein the system is configured to minimize the divergence using a metric that measures the divergence between a distribution of the virtual tokens and a distribution of input tokens.

Aspect (15): In an aspect (15), the present invention provides the method according to a tangible, non-transitory computer-readable medium having instructions thereon which, upon being executed by one or more hardware processors, alone or in combination, provide for execution of the steps of determining an information loss term for a set neural network that internally uses virtual tokens such that the information loss term minimizes a divergence between two distributions, and training the set neural network with training data from a data source that is expressed as sets using the information loss term.

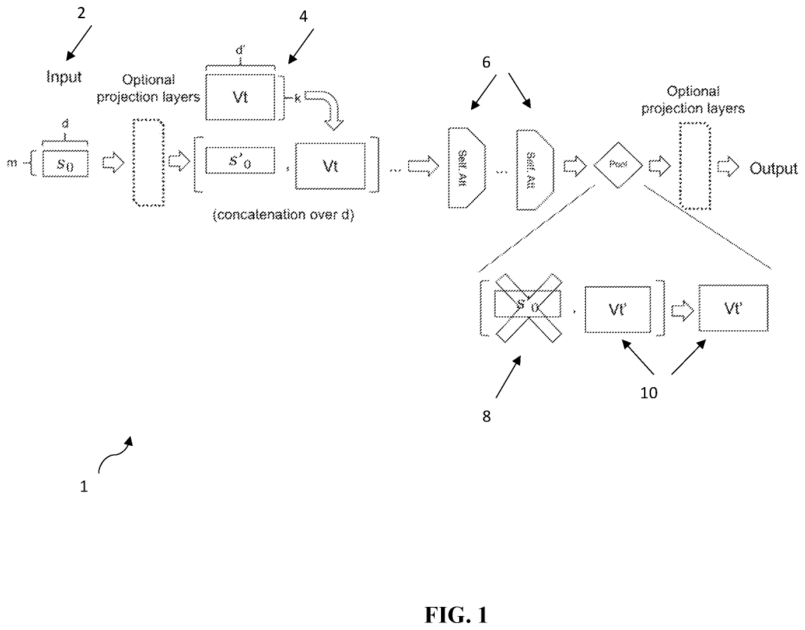

FIG. 1 shows an example diagram of a set neural network or transformer 1. The input 2 consists of a set of points with dimensions. The input 2 can be projected in a dimensional space. Then, the projected input set is concatenated with the virtual token set 4. This new batch is now fed to self-attention layers 6 and is going to be merged and projected again. After the batch is merged by the self-attention layers 6, the projection related to input 8 is removed, and virtual token projections 10 are taken. Finally, additional layers (e.g., dense layers) can be added, so the model can be adapted to the desired output.

Set neural networks are a sub-type of artificial neural networks that work with sets. This means, each sample of the dataset is a collection of datapoints Sᵢ={x₀, x₁, . . . xₘ} for x ∈ [symbol] ^{d}, where m is the number of datapoints of the set Sᵢ, d is the dimensionality of each datapoint, and the neural network model is permutation invariant. This implies that a set neural network is a function ƒ(·|Θ) where Θ are the neural networks parameters that, for a given input Sᵢ, keep its prediction invariant, no matter what is the arrangement of each data point x in S.

f(Sᵢ={x₃,x₂, . . . ,xₘ}|Θ)=f(Sᵢ={x₀,x₁,xₘ}|Θ)= . . . Formula (1)

An exemplary embodiment utilizes a transformer model with virtual tokens (Vt of size k×d′) as a reference model. In FIG. 1 a diagram of the exemplary transformer model is shown. However, the present invention is not tied to the particular model of FIG. 1, which uses special tokens that the model learns and uses to perform different tasks. Similar models to FIG. 1 are found in the special classification tokens, or “CLS” tokens, of the BERT model in Devlin, or a fixed collection of tokens as in Lee. BERT appears as a sentence classier, so in the context of Devlin, only a single virtual token was needed. Then, the BERT model of Devlin appears as an extension of BERT for point-cloud data. In order to efficiently encode this type of data, a single virtual token is not enough. Therefore, in Devlin they proposed to make use of a fixed collection of virtual tokens. These virtual tokens are landmarks on the latent space that are used to encode arbitrary sets.

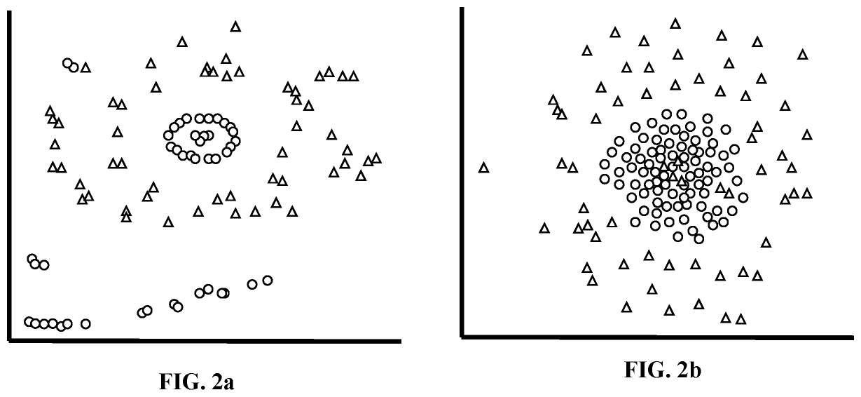

However, during the training, the distribution of the learned virtual tokens and the distribution of the input tokens are different. Even if the model can work, the distribution mismatch may cause an imperfect encoding. Experiments of a plain model observed the case of the FIG. 2a, where the distribution of the encoding is visualized using tSNE on a 2 dimension plot. Further embodiments of the present invention can produce the distribution in multiple dimensions. For example, if a projection layer is provided after the input, the dimensionality of the output can be created with chosen parameters. Or the dimensionality of the output can simply be the same dimensionality of the input, such as if no projection layer is provided. In an exemplary embodiment, a four dimensional vector [x, y, sin(a), cos(a)] can be made from an originally 3D input that is converted into polar coordinates.

FIGS. 2a and 2b are a tSNE projection of the virtual tokens and the input tokens of real input example of a fingerprint dataset. The virtual tokens that an exemplary model of the present invention learned are depicted as triangles, and the input tokens are depicted as circles. The plot of FIG. 2a shows the projection that is typically learned with the model described in FIG. 1. The plot of FIG. 2b shows the presentation that is achieved by the proposed invention.

FIG. 2a clearly shows how the input (circular) tokens and the virtual tokens (triangular) follow totally different distributions. Therefore, despite the great capability of the neural networks in learning to encode representations, there is already observed information loss.

A preferred embodiment of the present invention builds a mechanism that reduces, during the training, the information lost (i.e. the difference of the distributions between the virtual tokens and the encoding of the input points, or the number of “bits” that are missing in the virtual token space from the input space) in the encoding between the virtual tokens and the input tokens.

An exemplary embodiment of the present invention is based on information theory. Considering the input tokens as the true distribution, and the virtual tokens as the approximation distribution, the number of bits that are missing in the virtual token space can be measured to fully approximate the input token space. A Kullback-Leibler divergence is helpful in performing the measurement of the number of missing bits. Considering both distributions as Gaussian, the virtual token regularization loss can be defined as:

$${ℒ}_{Vt} ( {T}_{i n} \vert {T}_{Vt} ) = K L ( {T}_{i n} \vert \vert {T}_{Vt} ) = - \int {T}_{Vt} ( x ) log {T}_{i n} ( x ) dx + \int {T}_{Vt} ( x ) log {T}_{Vt} ( x ) dx = \frac{1}{2} log ( 2 {πσ}_{V t}^{2} ) + \frac{{σ}_{i n}^{2} + {( {μ}_{i n} + {μ}_{V t} )}^{2}}{2 {σ}_{V t}^{2}} - \frac{1}{2} ( 1 + log 2 {πσ}_{i n}^{2} ) = log \frac{{σ}_{V t}}{{σ}_{i n}} + \frac{{σ}_{i n}^{2} + {( {μ}_{i n} + {μ}_{V t} )}^{2}}{2 {σ}_{V t}^{2}} - \frac{1}{2} \qquad Formula ( 2 )$$

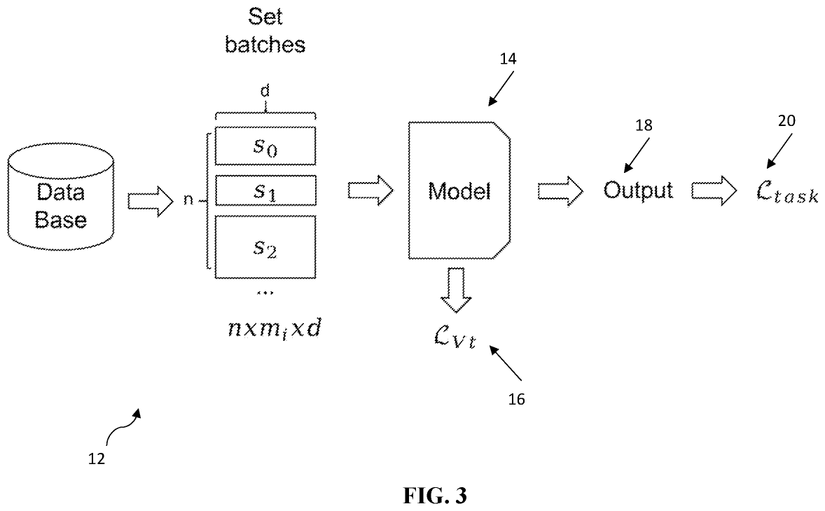

FIG. 3 shows a diagrammatic view 12 of an exemplary embodiment of the present invention, illustrating how an input is fed into the set neural network model 14 and how the losses are connected during the training time. The input set is mixed with the virtual tokens by the set neural network. During the mixture, the information loss term [symbol] _{Vt} 16 is computed. The model processes the input, and produces the desired output 18. The output is used to calculate the task specific loss [symbol] ₜₐₛₖ 20. The total loss can be computed as a weighted sum of the task specific loss [symbol] ₜₐₛₖ 20, the information loss term [symbol] _{Vt} 16, and, optionally, the other auxiliary losses:

[symbol]

=α [symbol] ₜₐₛₖ+β [symbol] _{Vt}+γ [symbol] ₐᵤₓ Formula (3)

where α,β, and γ are weights.

With the proposed loss L, the neural network can be trained with any conventional algorithm. During training, all the losses, [symbol] ₜₐₛₖ, [symbol] _{Vt}, [symbol] ₐᵤₓ can be used to minimize L, individually or in summation. Training aims to minimize [symbol] such that the virtual tokens become similar to the input tokens. After the training, the model can deployed. With the proposed loss [symbol] , training time is substantially reduced and the generalization capabilities of the resulting model are improved, as evidenced by the experimental results.

An exemplary training process can compute the gradient with respect to all losses and apply the gradient to the model parameters. This can be done by feeding a batch to the neural network, computing the losses for that batch, acquiring gradients for that batch, and applying updates to the neural network based on the gradients for that batch. This batch may employ a validation set, e.g., a set of points used only to compute [symbol] . This process can include as many iterations or epochs as necessary. Many ways for determining the appropriate number of iterations exist, e.g., setting a fixed number of epochs or evaluating the performance convergence. For example, if determining the appropriate number of iterations includes evaluating the performance convergence, the change in loss each iteration is evaluated, and the training is completed once the loss term no longer changes from iteration to iteration.

Although an embodiment utilizing the Kullback-Leibler divergence is a preferred way to achieve the desired effect for proposed information loss term [symbol] _{Vt} 16, a metric that measures the divergence between distributions might achieve similar results. Therefore, metrics such as the Wasserstein or Jensen-Shannon divergence can be used as replacements to the formula (2) according to other embodiments of the present invention.

A second possible variant of the invention is variational encoding by sampling the virtual tokens. Therefore, instead of having a fixed collection of learned virtual tokens, the mean and the variance of the virtual tokens may be used, as are learned in formula (2) (i.e.: μ_{Vt} and σ_{Vt}) for sampling them from a Gaussian distribution with these parameters.

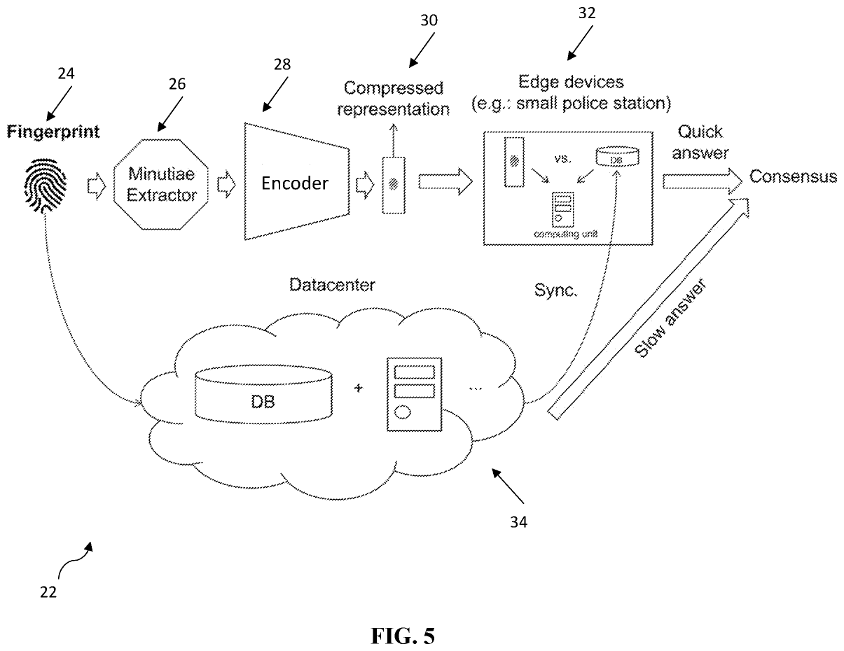

FIG. 5 discloses an exemplary system 22 of the present invention that provides for faster fingerprint matching through the training of a neural network to analyze the minutiae of fingerprints 24. Fingerprints 24 are impressions that are typically left on an object after touching them with the hands. These impressions contain patterns that are unique to the individual that left them. This is one of the reasons why they are massively collected by the security agencies of governments and stored in large datasets. The data is at the origin of an image, but it is commonly processed to extract the minutiae. The minutiae are the locations where two lines are joining or splitting. Additionally, the location of the angle at which two lines join can be added. This converts the fingerprint 24 into a set of minutiae. The exemplary embodiment of the present invention efficiently encodes in a compressed representation 30 the sets of minutiae of the fingerprints 24. This compressed representation 30 substantially reduces the computation and memory space for carrying out the method. This allows the usage of conventional hardware and can open the door to the computation on the edge.

FIG. 5 outlines an exemplary embodiment of a fingerprint system 22. At first, the fingerprint 24 is obtained by a scanner, photography, or other sources. The raw data is sent to the datacenter 34. In the datacenter 34 the data will be stored, processed, and possible access could be sent. In parallel, the minutiae can be extracted by a minutiae extractor 26, and fed into an encoder 28 to be encoded and compressed into a compressed representation 30. The encoded representation can now be used in light stored devices and simple computing units, i.e., edge devices 32, which allow getting a quick and offline answer on the edge. Later on, the answer can be contrasted with the answer provided by the datacenter 34, and the edge database can be updated. Therefore, in an exemplary use according to the embodiment of FIG. 5, an edge device 32 can compare a fingerprint 24 obtained after training to the compressed representation 30, i.e., provide information to compare to the compressed representation 30. Or, as a further exemplary use, an edge device 32 can query the compressed representation 30 which has already performed matches of a set of fingerprints 24. As a still further exemplary use of FIG. 5, an edge device 32 can provide the compressed information 30, send it to the datacenter 34 that performs the query, and get the solution back.

Fingerprints 24 contain patterns that can be used as unique identifiers of an individual. Minutiae sets are the gold standard features that are used for the fingerprint matching task. This task needs to compute similarities of sets of different sizes and has to be done for millions of individuals, making the task challenging and computationally expensive. In an embodiment of the present invention, an AI-driven solution is presented with a mechanism that minimizes information loss during the encoding of minutiae sets. In lab experiments, a 99.9% accuracy was achieved, as well as a speed of more than ten billion matches per second.

The exemplary embodiment of the present invention is tested on a private dataset of minutiae from fingerprints. Generally, testing can include comparing the compressed representations. The inputs are supplied as in the training phase, but they do not necessarily have to be the training datasets, as the inputs can be new datasets. In this embodiment, each sample of the dataset is a set of minutiae locations, and the angles that were extracted from fingerprints 24. The model is trained to optimize the similarity between distorted versions of the same sample. In other words, [symbol] ₜₐₛₖ= [symbol] _{contrastive}(s₀, s₀′) [symbol] _{Vt} 16 is exactly as previously described.

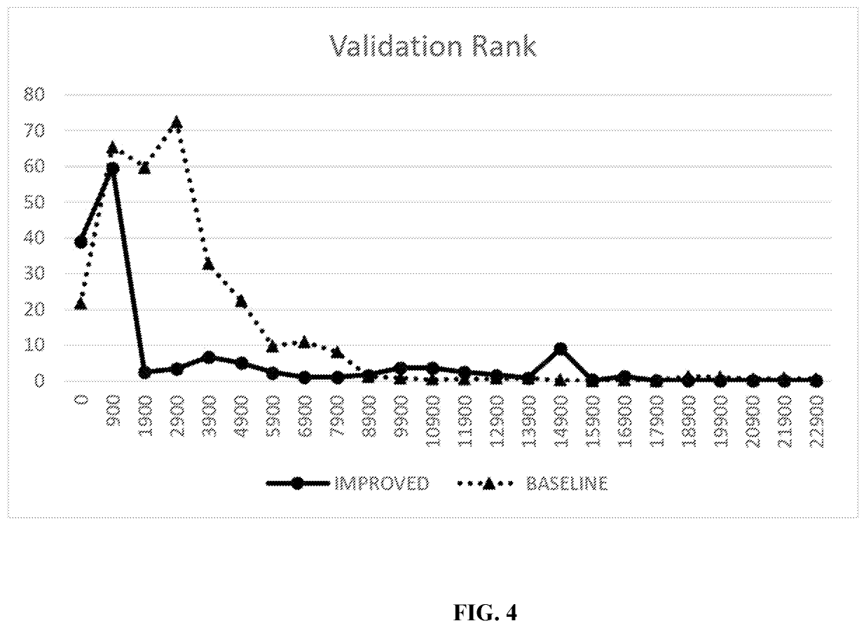

FIG. 4 ranks progress of the validation over time during the training, comparing the results of an exemplary embodiment of the present invention and a baseline model without the improvements of the present invention. The baseline model is represented with a dashed line and triangular nodes, and the resulting model of an exemplary embodiment of the present invention is represented with a continuous line and circular nodes. The depicted rank is the mean position value in which the correct matches were found, and so the first position is the best value for the rank. FIG. 4 shows how the ranking over the validation set is improved much faster than the baseline without the proposed [symbol] _{Vt}. Apart from a faster training, the results of the exemplary embodiment of the present invention shows improvements over the results on the test set in the following Table 1.

TABLE 1

| Metric | Baseline | Improved |
|---|---|---|
| Mean rank | 0.174999997 | 0.030999999 |
| Std rank | 1.687120438 | 0.178994402 |
| Max rank | 44 | 2 |
| Hit@ 1 rank | 0.976999998 | 0.999000013 |
| Hit@ 10 rank | 0.996999979 | 1 |

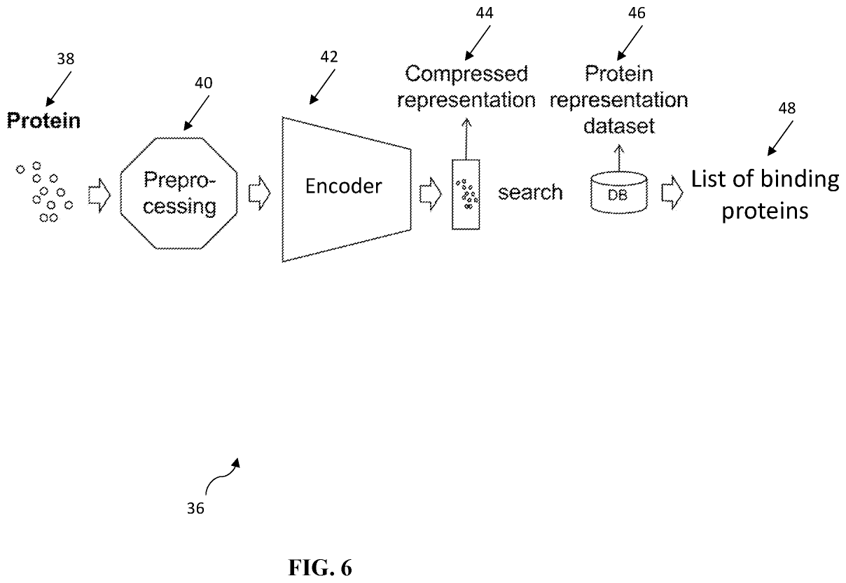

FIG. 6 discloses an exemplary embodiment that provides for modeling protein binding interactions 36 on vaccines. Protein molecules 38 are clouds of interconnected atoms that form large complex structures. For example, each atom within a protein molecule 38 will have a relative three dimensional location within the protein molecule 38 and the features associated to the chemical elements which, when mapped to (x,y,z), can yield a set representation of the molecule. The biomedical function of the protein molecules 38 is typically defined by the 3D shape of the surface of the molecule 38. Therefore, by depicting a molecule 38 as a set of 3D points and its associated features, the exemplary embodiment of the present invention can learn a model that outputs predictions on the protein-to-protein binding. That model can find within a dataset of proteins a list of binding candidates 48 by matching a compressed representation 44 of the molecule 38 with a precomputed compressed representation of the other proteins of a dataset. In an embodiment, the data obtained regarding the protein molecules 38 undergoes pre-processing 40, and an encoder 42 creates a compressed representation 44, that, in combination with a protein representation dataset 46, can find within a dataset of proteins a list of binding candidates 48.

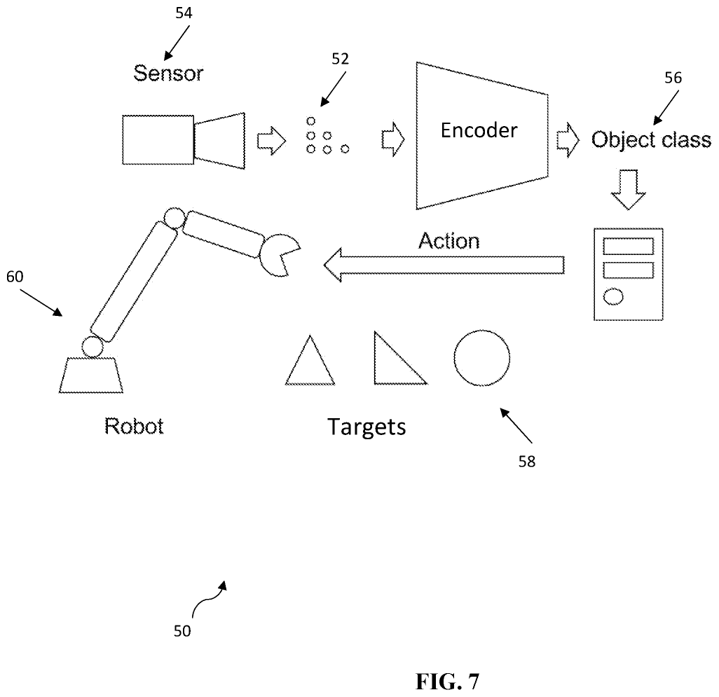

FIG. 7 discloses one exemplary embodiment of the present invention that provides for point-cloud object classification 50 in the field of robotics. In many fields such as robotics, autonomous navigation, or augmented reality (among others), point clouds represent an important source of geometric information about the world, where an object, such as a target 58, is typically defined by a set of 3D points 52 that are obtained by a sensor 54 such as a laser scanner. Therefore, being able to classify objects from 3D point clouds 52 into an object class 56 plays an important role in these fields. Similarly, as in the previous cases, an embodiment of the present invention can be used to efficiently learn representations of the point cloud datasets that can be used to solve a task specific to the robotic goal. As an example, an embodiment of the present invention can be applied to the situation in which a robotic arm 60 has to pick the desired object, i.e., targets 58, to interact with it.

Embodiments of the present provide the following improvements and advantages over existing approaches:
1. Using [symbol] _{Vt} as a mechanism that minimizes the information lost of the encoding performed by the virtual tokens and the input during the training in a set neural network. The proposed mechanism utilizes the minimization of the divergence between two distributions with metrics such as KL divergence (preferably), or others.
2. As compared to a baseline model, embodiments of the present invention provide improvements in the training time and the performance of the model.
3. Providing a direct impact to increase the convergence speed during the training, to increase the generalization capability of the model, and to improve the final performance of the model.
4. Conserving and providing reductions in computational resources and costs and a reduction in the energy that is required for training the model.
5. Reducing the need for retraining the model due to better generalization.
6. Providing for a superior performance of the final system.

An embodiment of the present invention provides a method for efficiently training set neural networks, wherein the method comprises the steps of:
1 Obtaining a data source that can be expressed as sets;
2. Providing a training dataset and a set neural network that internally uses virtual tokens.
3. Training the neural network with the proposed information loss term [symbol] _{Vt} for the minimization of the divergence between two distributions with metrics such as KL divergence (preferably), or others.
4. Testing and deploying the method.

Embodiments of the present invention may have advantageous direct application on fingerprint technologies. However, the present invention is not limited to fingerprint technologies, and embodiments could be applied to improve other technical areas of exploitation such as protein binding interactions on vaccines. Embodiments of the invention can be advantageously applied to domains in which the data can be expressed as sets, and to set models that internally make use of collections of virtual tokens.

While subject matter of the present disclosure has been illustrated and described in detail in the drawings and foregoing description, such illustration and description are to be considered illustrative or exemplary and not restrictive. Any statement made herein characterizing the invention is also to be considered illustrative or exemplary and not restrictive as the invention is defined by the claims. It will be understood that changes and modifications may be made, by those of ordinary skill in the art, within the scope of the following claims, which may include any combination of features from different embodiments described above.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

What is claimed is:

1. A method for minimizing information loss in set neural networks, the method comprising:
determining an information loss term for a set neural network that internally uses virtual tokens such that the information loss term minimizes a divergence between two distributions; and
training the set neural network with training data from a data source that is expressed as sets using the information loss term.

2. The method of claim 1, wherein minimizing the divergence is performed using a metric that measures the divergence between a distribution of the virtual tokens and a distribution of input tokens.

3. The method of claim 2, wherein the metric is a Kullback-Leibler divergence, Wasserstein, or Jensen-Shannon divergence.

4. The method of claim 1, further comprising testing the trained set neural network.

5. The method of claim 1, further comprising using the trained set neural network to produce a compressed representation of input data in a machine learning task.

6. The method of claim 1, wherein the method further comprises:
obtaining minutiae of fingerprints as the training data from the data source that is expressed as sets; and
encoding in a compressed representation the minutiae of fingerprints.

7. The method of claim 6, wherein the method comprises:
using the compressed representation to match a fingerprint.

8. The method of claim 1, wherein the method further comprises:
depicting a protein molecule as a set of 3D points as the data source that can be expressed as sets; and
using the trained set neural network to predict a protein binding candidate from a protein representation dataset.

9. The method of claim 1, further comprising:
defining an object by a set of 3D points as the training data from the data source that is expressed as sets; and
using the trained set neural network to classify the object into an object class.

10. The method of claim 1, wherein the set neural network internally uses mean and variance of the virtual tokens during the training.

11. The method of claim 1, further comprising encoding a compressed representation of data of the data source that is expressed as sets using the trained set neural network.

12. The method of claim 1, wherein the divergence is between a distribution of virtual tokens and a distribution of input tokens and the divergence approximates an input token space.

13. A system comprising one or more hardware processors which, alone or in combination, are configured to provide for execution of the following steps:
determining an information loss term for a set neural network that internally uses virtual tokens such that the information loss term minimizes a divergence between two distributions; and
training the set neural network with training data from a data source that is expressed as sets using the information loss term.

14. The system of claim 13, wherein the system is configured to minimize the divergence using a metric that measures the divergence between a distribution of the virtual tokens and a distribution of input tokens.

15. A tangible, non-transitory computer-readable medium having instructions thereon which, upon being executed by one or more hardware processors, alone or in combination, provide for execution of the following steps:
determining an information loss term for a set neural network that internally uses virtual tokens such that the information loss term minimizes a divergence between two distributions; and
training the set neural network with training data from a data source that is expressed as sets using the information loss term.
